(19) United States
(12) Patent Application Publication
(10) Pub. No.: US 2013/0078641 A1
(43) Pub. Date: Mar. 28, 2013

(54) SAMPLE PROCESSING METHOD AND DEVICE

(71) Applicants: Hendrik J. Viljoen, Lincoln, NE (US); Scott E. Whitney, Lincoln, NE (US); Alison Freifeld, Omaha, NE (US); Christine Booth, Lincoln, NE (US); Xing Zhao, Omaha, NE (US)

(72) Inventors: Hendrik J. Viljoen, Lincoln, NE (US); Scott E. Whitney, Lincoln, NE (US); Alison Freifeld, Omaha, NE (US); Christine Booth, Lincoln, NE (US); Xing Zhao, Omaha, NE (US)

(73) Assignee: STRECK, INC., Lavista, NE (US)

(21) Appl. No.: 13/700,817

(22) PCT Filed: Jun. 1, 2011
(86) PCT No.: PCT/US11/38766

§ 371 Date: Nov. 29, 2012

Related U.S. Application Data
(60) Provisional application No. 61/350,057, filed on Jun. 1, 2010.

(30) Foreign Application Priority Data
Jun. 1, 2011 (US) PCT/US2011/038766

(51) Int. Cl. C12Q 1/68

(52) U.S. Cl. CPC C12Q 1/6806
USPC 435/6.12

(57) ABSTRACT

The present invention provides a method and device for treating and analyzing a biological specimen. The biological specimen is introduced into a processing device and treated thermally, mechanically, chemically or any combination thereof within the processing device to alter at least one constitutive characteristic of the biological specimen and to release or create one or more biological indicators from the biological specimen. The biological specimen is further contacted with a treated polymeric material so that at least a portion of the polymeric material binds to the one or more biological indicators.

### CLAIM OF PRIORITY

This application claims the benefit of the filing date of U.S. Provisional Application Ser. No. 61/350,057 filed on Jun. 1, 2010, the entirety of the contents of that application being hereby expressly incorporated by reference.

### FIELD OF THE INVENTION

The teachings herein relate to the processing and analysis of a biological specimen.

### BACKGROUND OF THE INVENTION

The processing and analysis of biological specimens presents a number of challenges, especially in determining the biological indicators that may be isolated and analyzed for accurate information regarding the absence or presence of a particular gene sequence and/or disease. Many commonly used diagnostic tests result in false positives and false negatives, or provide results that require additional testing for verification. These inaccuracies and additional tests result in increased time and cost associated with accurate diagnosis. As such, it may be necessary to develop more sensitive and refined procedures for treating and analyzing a biological specimen so that the time and cost associated with accurate diagnosis can be reduced. This is especially true in underdeveloped areas of the world where certain technology required for disease diagnosis is unavailable and many infected individuals cannot afford the costs for testing. The time for diagnosis may also be of concern in these areas as infected individuals may travel many miles to seek diagnosis but are unable to remain at a test site for extended periods to receive their diagnosis.

Notwithstanding the above, there remains a need for biological specimen treatment protocols that effectively prepare a specimen so that the necessary biological indicators can be isolated without damage. There is also a need for specimen isolation and analysis protocols that allow for quick, low-cost, and accurate disease diagnosis.

The present teachings address the above needs by providing a point-of-care diagnostic device that includes components for treating a biological specimen so that one or more biological indicators are isolated from the specimen. The present teachings further provide for analysis of the one or more isolated biological indicators so that selected forms of a disease are identified.

### SUMMARY OF THE INVENTION

The present teachings provide a method for introducing a biological specimen into a processing device, treating the biological specimen thermally, mechanically, chemically or any combination thereof within the processing device to alter at least one constitutive characteristic of the biological specimen. The present teachings further provide a method for the release and/or creation of one or more biological indicators from the biological specimen and contacting the treated biological specimen with a treated polymeric material (e.g., a capture strip) so that at least a portion of the polymeric material binds to the one or more biological indicators.

As referred to herein, constitutive characteristics of a biological specimen may include one or more characteristics of the biological specimen, and may include a physical characteristic, a chemical characteristic, or both. It may include one or more of a composition, a concentration, a chemical reaction, a mechanical characteristic, a morphological characteristic, a rheological characteristic, an electrical characteristic, an optical characteristic, a magnetic characteristic, a thermal characteristic, or any combination thereof. Any altering of a constitutive characteristic may be irreversible, or alternatively, a biological specimen may undergo additional treatment to reverse or modify the alteration of a constitutive characteristic in the context of a biological specimen having an increased viscosity, the material may be processed for altering one or more rheological characteristics.

The present teachings further provide for a processing device (e.g., a lysis micro-reactor or LMR) for use with biological specimens comprising a mixing portion, a capture means, at least one interface for a control device and a covering means. The capture means may include a polymeric material located therein for attaching to one or more biological indicators. The processing device may also include a processing well, a fluid transport path, at least one heating element, a temperature sensing device and a covering. The processing device may also include a cooling device. The processing well may be adapted to receive a device for mixing and pumping a biological specimen. The fluid transport path may include a valve. The at least one heating element may he disposed proximate the processing well. The temperature sensing device may be disposed proximate the processing well. The covering may be placed over the processing well so that the contents of the processing well remain within the body. Examples of suitable processing devices may be found in U.S. application Ser. No. 12/780,345, filed on May 14, 2010 and incorporated by reference herein for all purposes.

The biological specimen may also be collected in a specialized specimen container that reduces the risk of contact with the specimen for medical professionals. Examples of suitable specimen containers may be found in U.S. application Ser. No. 12/780,508, filed on May 14, 2010 and incorporated by reference herein for ail purposes.

The teachings herein contemplate a device and method for the treatment and analysis of a biological specimen so that multiple biological indicators may be simultaneously identified. A biological specimen may be treated so that target biological indicators are not damaged and are able to be isolated from the remainder of the biological specimen. The biological specimen may be treated and/or analyzed in a processing device. The processing device may include one or more portions of an adherent material that attracts one or more target biological indicators. The adherent material may include a polymeric material. The target biological indicators may adhere to the adherent material so that the biological indicators are isolated and later analyzed. The analysis may include a step of amplifying the target biological indicators.

### BRIEF DESCRIPTION OF THE DRAWINGS

FIG. 1 is an illustrative example of a capture strip in accordance with the present teachings.

FIG. 2 is an illustrative example of a lysis micro-reactor in accordance with the present teachings.

FIG. 3 is an illustrative example of the transfer of the capture strip from the lysis micro-reactor to a cuvette in accordance with the present teachings.

### DETAILED DESCRIPTION

In general, the teachings herein contemplate a method and device for the treatment of a biological specimen, and the collection and analysis of biological indicators located therein. The processing equipment disclosed herein allows for simultaneous treatment of a biological specimen and isolation and collection of one or more target biological indicators therein. The treatment may occur so that the biological specimen releases or creates a target biological indicator for analysis. For example, a lysing step may be employed by which a cell well or cell membrane is degraded to release one or more nucleic acids and/or proteins contained therein. Complete processing, amplification and/or analysis may occur in a shortened time frame (e.g., less than about 5 hours, less than about 2 hours, less than about 1 hour, or even less than about 0.5 hours) For example, patients can provide a sample and receive a diagnosis in one trip to a health care facility. The present teachings have particular applicability and are used for testing biological specimens for diagnosing a disease and/or drug-resistant strains of a disease, or any other health condition. As an example, the present teachings may be used to detect multiple drug-resistant tuberculosis (MDR-TB), extensively drug-resistant tuberculosis (XDR-TB), Clostridium difficile, Clostridium perfringens, methicillin-resistant staph aureus (MRSA), vancomycin intermediate staph aureus (VISA), vancomycin-resistant staph aureus (VRSA), or the like.

More particularly, the present teachings provide a processing device, also referred to herein as a lysis micro-reactor (LMR). The processing device may include a processing well having an adherent material therein, also referred to herein as a capture strip. The processing device may receive a biological specimen in the processing well where the biological specimen is treated mechanically, thermally and/or chemically. The treatment may result in isolation of one or more target biological indicators. The one or more biological indicators may adhere to the adherent material within the processing well The biological specimen may be subsequently transferred via the adherent material (to a location within or external to the processing device) and amplified. The treatment of the biological specimen may include chemical modification of the biological specimen's rheology to promote flow and mixing, lysis of cells to release DNA, RNA, proteins and/or antigens, reduction of reaction (e.g., PCR) inhibitors from the biological specimen and/or transfer of the biological specimen or a portion of the biological specimen to an amplification and/or detection location.

The biological specimen may include blood, saliva, sputum, tissue, feces, urine, semen, vaginal secretions, hair, tears, biopsy material, cerebral fluid, spinal fluid, bone material or any other biological or chemical sample that may be tested for disease presence.

The mechanical processing may include a mixing member and motor for mixing a biological specimen in the processing well (e.g., mixing well). The thermal processing may include active temperature control (e.g., active heating and/or active cooling via fan and/or peltier device) of the biological specimen to one or more elevated and/or lowered temperatures. The chemical processing may include contacting the biological specimen with one or more chemical agents. Each of the mechanical, thermal and/or chemical processing steps may modify the biological specimen so that the biological specimen or a portion of the biological specimen is formatted for accurate analysis. The formatting process may include steps to reduce the viscosity of a biological specimen, lyse the cells within a biological specimen, protect the cells from unwanted nuclease and/or protease effects, adhere a portion of the biological specimen to an adherent material located within the processing device, or any other treatment so that any eventual analysis of the biological specimen or a portion of the biological specimen will be facilitated and/or improved (e.g., by resolving inconsistencies with the composition of the biological specimen). The mechanical, chemical, and/or thermal treatment may cause a biological specimen to release or create one or more target biological indicators that may be contained within the biological specimen prior to treatment. Each of the mechanical, chemical and/or thermal treatment steps may assist in extracting the one or more target biological indicators from the biological specimen. The target biological indicator may include DNA, RNA, proteins, antigens, serum, cells, plasma, contaminants, reaction products, hybridization targets, or any combination thereof.

As discussed above, it is possible that the mixing well may include, be composed of or contacted/coated with an adherent material that attracts and/or captures the target biological indicators. The adherent material may include or be composed of a filter, chromatography column, hybridization area, plastic, glass, at least one bead, immobilized DNA/RNA probe, immobilized antibody, an optical microarray device, or any combination thereof. The adherent material may he a polymeric strip. The mechanical processing (e.g., mixing) may cause sufficient turbulence to contact the target biological indicators to the well itself or the adherent material. The adherent material and any target biological indicators attached to the adherent material may then be removed from the mixing well and transferred to an amplification portion. The adherent material may be transferred by a transport means to an amplification portion or may be amplified within the transport means. The transfer of the adherent material may be facilitated by a force or means for pulling the material through the transport means. Alternatively, the material may remain within the mixing well with the target biological indicator attached thereto and the remaining biological specimen may be pumped out of or removed from the mixing well, thus allowing amplification to fake place within the mixing well. A wash step may be incorporated to remove any remaining biological specimen from the target biological indicator.

It is possible that the adherent material may be composed of a polymeric material. The polymeric material may include thermoplastics, thermoset plastics, elastomeric containing materials or any combination thereof. Examples of polymeric and elastomeric materials that may be employed include PTFE, PEEK, delrin, nylon, polyvinyl chloride, polypropylene, high-density polyethylene, low-density polyethylene, linear low-density polyethylene, polyvinylidene chloride polyamide, polyester, polystyrene, polyethylene, polyethylene terephthlate, bio-based plastics/biopolymers (e.g., poly lactic acid), silicone, acrylonitrile butadiene styrene (ABS), rubber, polyisoprene, butyl rubber, polybutadiene, EPM rubber, EPDM rubber, or any combination thereof.

One or more of the target biological indicators may occur naturally in a patient, making it challenging to identify biological indicators that may be causing disease and those which are not. It may thus be necessary to identify biological indicators that may cooperate with other biological indicators to result in a symptomatic disease state within a patient. As an example, the one or more biological indicators may include a toxin The toxin may cooperate with a bacteria, virus and/or particular genetic sequence and thus may be indicative of the presence of a disease or condition. The toxin atone or the bacteria, virus or genetic sequence alone may be naturally occurring in a patient and may thus cause no disease related symptoms. A toxin may be identified and isolated along with an associated bacteria, virus and/or particular genetic sequence such that the presence of both may be indicative of a symptomatic disease state. Therefore it may be necessary to test for both the genetic presence of a bacteria, virus and/or genetic sequence and the presence of an expressed toxin to make an accurate diagnosis.

In order to effectively identify the presence of a symptomatic disease or condition only (so that asymptomatic disease does not produce a positive test result), it may be necessary to develop treatment and isolation protocols that simultaneously selectively isolate multiple biological indicators from one biological specimen. As an example, a biological specimen may be treated and/or contacted so that a biological indicator associated with a particular bacteria is isolated along with a toxin that, when present in a patient along with the bacteria biological indicator, produces a symptomatic disease state.

It is possible that the capture strip may contact a biological specimen so that the capture strip is treated to attract multiple specific biological indicators. In the case of the bacteria and associated toxin discussed above, the adherent material must he treated to attract the DNA (or other biological indicator) of the bacteria while also attracting an antigen (or other biological indicator) relating to the toxin. The adherent material may be treated so as to attract only these specific biological indicators, leaving any remaining biological specimen unattached to the adherent material so that only the biological indicators attached to the adherent material are isolated.

It is further possible that any isolated biological indicators will be subjected to further analysis. The biological indicators may be analyzed by a PCR-based test. It may therefore be necessary to include treatment steps so that each biological indicator may be successfully amplified by the PCR process. As an example, it may be necessary to treat a biological specimen so that probes, tags or other identifiers are attached to specified biological indicators. These identifiers may be capable of being amplified via a PCR process in the event that the biological indicators themselves are not.

It is possible that a biological specimen may contain inhibitors that interfere with PCR reactions. Thus, specimens may undergo extensive mechanical, chemical, and or thermal treatment. Treatment may involve a step of cell lysis followed by binding of DNA from within the lysed cells. This bound, concentrated DNA may then he easily removed from the remainder of the specimen containing the inhibitors.

The adherent material (e.g., capture strips) may be chemically and/or structurally pre-treated to improve the adhesion characteristics of the material and the specificity with which the material only adheres to certain desired biological indicators. The capture strips may be contacted with a detergent The detergent may be selected from the group consisting of N-(3-dimethylaminopropyl)-N′-ethylcarbodiimide hydrochloride) (EDC), sodium lauryl sulfate, cetrimonium bromide, polyoxyethylene glycol, 3-[(3-cholamidopropyl)dimethylammonio]-1-propanesulfonate (CHAPS), glycosides (i.e. octyl-thio-glucoside, maltosides), phosphine oxides, or any combination thereof. The capture strips may be washed with one or more of Tris-HCl, NaCl, Tween 20, or combinations thereof. The capture strips may be washed with de-ionized water and stored in de-ionized water until use. The capture strips may further be contacted with one or more oligomers and/or polymers to further promote adhesion between the adherent material and the biological indicators. The capture strips may also undergo surface treatment. The capture strips may be sanded so as to provide texture to the adherent material thus improving adhesion between the adherent material and the biological indicators.

The processing of a biological specimen may involve the following steps. One or more capture strips (e.g., an adherent material) may be placed in a lysis micro-reactor (e.g., mixing well) as described herein. A biological specimen and lysis buffer may then be added to the mixing well. One or more chelating beads/resin powders may be added to the mixing well. One or more antibody labeled DNA templates (e.g., a DNA tag) may also be added to the mixing well. The substrate may then be incubated at from about 50° C. to about 85° C. with gentle stirring. The temperature of the biological specimen may then be increased to from about 60° C. to about 120° C. and a more aggressive mixing protocol may be followed to facilitate cell lysis. The capture strip may then he removed and washed.

The capture strips that contain the immobilized bacterial DNA may then be placed in a PCR cuvette with a master mix that contains primer sets for the bacterial DNA sequence. As an example, the capture strip containing any target biological indicator may be located into the device disclosed in U.S. Provisional Application No. 61/477,785, tiled Apr. 21, 2011 and incorporated by reference herein for all purposes. The products can be detected either in real time with molecular beacons or end point determinations can be made by gel electrophoresis. Parallel amplification of two targets may constitute the biochemical AND gate. Thus, two PCR products may be used to indicate the presence of both the bacteria and any associated toxin.

As stated above, the mechanical processing may include the use of a mixing member located in a mixing well (e.g., processing well, mixing portion, or lysis micro-reactor). The mixing member may include an impeller structure that facilitates both mixing of a biological specimen in the well and pumping of the biological specimen or a portion of the biological specimen out of the well. The mixing member may cyclically impinge upon the biological specimen to alter or facilitate alteration of at least one constitutive characteristic of the biological specimen. The mixing member may be reciprocally activated, rotationally activated or a combination thereof through a number of cycles. The mixing member may promote contact between the biological specimen and the capture strip.

A motor may be included to cause the mixing member to spin, oscillate, cycle or have any similar motion that imparts shear to a biological specimen, causes turbulence within a biological specimen, or both. The mixing member may be an impeller structure having a shaft portion that contacts or nearly contacts at least one wall of the mixing well. For example, a sleeve and/or bearing may be present between the wall and the shaft such that the shaft may rotate freely while providing a fluid tight seal. Alternatively, the shaft may contact or almost contact the mixing well through the covering means. For example, the shaft portion may be integrated into a hinged lid. The motor may include an output shaft that engages an input shaft of the mixing member or mixing well. The spinning of the mixing member and any attached shafts or structural members may promote increased mixing and contacting rates with any chemical agents located within the well. The spinning may also reduce the viscosity of the biological specimen and may accelerate any hybridization reactions within the mixing well (such as DNA, RNA or protein hybridization to probes or affinity media). The motor may be activated by a controller which may control the torque and/or direction of the mixing member. The desired torque of the mixing member may be driven by the viscosity of the biological specimen. Advantageously, a biological specimen having a higher viscosity may be mixed at a higher torque to effectively break down the biological specimen. A biological specimen having a lower viscosity may be mixed at a lower torque as the break down process is minimized. The mixing member may be a magnetized impeller that is activated by magnetic field manipulation proximate the mixing well. The movement (e.g., spinning) of the mixing member may impart heat to a biological specimen.

The function of the mixer enables the DMA capturing capability of the capture strip. The mixing allows for transfer of DNA from the biological sample to the PCR cuvette at copy numbers as low as 31 copies/ml sample which is equivalent to 0.05 ato-molar. The high efficiency of the capture strip may be attributed to the fluid flow in the processing device. The impeller may create rotational flow that varies with radial position. The maximum speed may thus correspond to the impeller radius. Therefore, at the wall of the processing device and at the capture strip surface, the velocities may be zero. The capture strip may be formed as a rectangle or cylinder so that at least a portion of the polymeric strip is parallel to the axis of the processing device. The maximum value of the rotational velocity may drop-off towards the wall of the processing device and towards the capture strip. The DNA molecules may have primarily rotational flow and therefore, a DNA molecule may not stay on the same trajectory every rotation. The capture strip may exert an electrostatic force on the DNA that pulls the molecules across streamlines. The overall motion is thus determined by the combination of the electrostatic force, which decays rapidly as the distance to the capture strip increases. At higher impeller speeds, such as 50 revolutions per second, ONA molecules may be swept past the strip if their speed is too high. An impeller speed of about 25 rotations per minute may be more likely to allow for the DNA to be captured on the capture strip.

In addition to the impeller, the processing device may also include a pumping mechanism for pumping all or a portion of a biological specimen out of the mixing well. The pumping mechanism may also include a structural member that may be the mixing member The structural member may move in one direction (e.g., counter-clockwise) for mixing purposes and the opposite direction (e.g., clockwise) for pumping purposes. The pumping mechanism may also employ pressure gradients to assist the biological specimen in moving into and/or out of the mixing well. The pumping mechanism may pump the biological specimen or a portion of the biological specimen into an amplification well. A detection well may be similarly used or the detection step integrated into the amplification well (e.g., by real-time PCR). The pumping mechanism may pump the biological specimen or a portion of the biological specimen through a transport path to the amplification well. The biological specimen or a portion of the biological specimen may be transported from the mixing well to another location by capillary forces (e.g., by wicking).

The pumping mechanism may pump the biological specimen or a portion of the biological specimen through a transport path. Amplification and/or detection may occur in the transport path, thus removing the need for an amplification well. In the event that amplification and/or detection occurs in the transport path, a waste well may collect any remaining biological specimen after the biological specimen has undergone amplification and/or detection in the transport path. It may also be possible that the processing device includes a well for DNA amplification that is downstream of the mixing well so that the biological specimen is transported via the transport path from the mixing well to the well for DNA amplification. Further, a separate well for reverse transcription of RNA may be included within the processing device, or external of the processing device. The mixing well may also include one or more entry and/or exit ports for the entry and exit of biological specimen, target biological indicator, chemical processing agents, or any combination thereof.

In addition to mechanical processing via mixing, the processing device may further process a biological specimen by contacting the biological specimen with one or more chemical processing agents. The chemical processing agents may be added to the biological specimen to prepare the biological specimen for amplification and/or detection and to cause the biological specimen to release or create a desired target biological indicator. The chemical processing agents may be added to the mixing well prior to addition of the biological specimen. The chemical processing agents may be added to the biological specimen prior to, during or after any mixing step. The chemical processing agents may be added to the biological specimen prior to, during or after any heating and or cooling step. Depending upon the composition of the biological specimen, the chemical processing agents contacted with the biological specimen may differ. It is possible that the chemical processing agents may be stored within the processing device. The chemical processing agents, which may be pre-sealed during manufacture, may be located in a reagent well or reservoir prior to contact with a biological specimen. The processing device may thus include a channel that transfers the chemical processing agents from the reagent well to the mixing well for treatment of a biological specimen. It is possible that the chemical processing agents may be pre-loaded within the mixing well prior to entry of the biological specimen into the mixing well. The chemical processing agents may also be contacted with the biological specimen or target biological indicator within the transport means. The chemical processing agents may include an additional mixer and/or pumping mechanism in the reagent well.

The chemical processing agents may include one or more of a variety of agents such that the selection of the appropriate agents will depend upon the composition of the biological specimen and the desired function of the agent within the biological specimen. For example, in the event that a biological specimen treatment protocol is performed so that a biological specimen releases DNA as a target biological indicator, the chemical processing agents may include a lysis buffer to promote cell lysis so that cellular DNA (the target biological indicator in this case) is released as a result of the cell lysis process. The type of chemical processing agents that may be used include but are not limited to reducing agents, nuclease inhibitors, enzymes, lysis buffers, protease inhibitors, phosphatase inhibitors, metabolic inhibitors, enzyme inhibitors, fixatives (e.g., protective agents), acids, bases, organic solvents, alcohols, drying agents, water, heavy water, mucolytic agents, sterilizers or any combination thereof. A nuclease inhibitor may also be present to protect the DNA from damage from any nucleases that may be present in the biological specimen.

In order to stimulate release or creation of a target biological indicator, it may be desirable to lyse cells located within the biological specimen. The methods herein may include one or more steps of stimulating release or creation of target biological indicator including one or more lysis steps. The lysing may include treating the biological specimen physically and/or thermally for rupturing a cell wall or membrane so that cell contents are expelled from within the cell. One approach contemplates chemically treating a biological specimen with an agent such as a lysis buffer. Examples of lysis buffers that may be used include but are not limited to tris-HCl, EDTA (ethylenediaminetetraacetic acid), tris-EDTA, EGTA, SDS, deoxycholate, TritonX, NaCl, sodium phosphate, NP-40, phosphate buffered saline (PBS) and combinations thereof. The lysis buffer may include one or any combination of TCEP (Tris[2-carboxyethyl]phosphine) and Tris-EDTA.

The concentration of lysis buffer for lysing cells within the biological specimen may be at least about 0.25 mM or even 5 mM. The concentration of lysis buffer may be less than about 30 mM or even 20 mM. The concentration of lysis buffer may be from about 1 mM to about 20 mM. As an example, the biological specimen may be contacted by a lysis buffer including from about 0.5 mM to about 5 mM EDTA. The lysis buffer may include from about 5 mM to about 15 mM Tris-HCS. The lysis buffer may include from about 10 mM to about 30 mM TCEP. The lysis buffer may include from about 0.5 mM to about 5 mM Tris-EDTA and from about 5 mM to about 20 mM TCEP at a concentration of at least about 20×. The lysis buffer may include from about 0.5 mM to about 5 mM EDTA and from about 5 mM to about 15 mM Tris-HCl at a concentration of less than about 100×. The lysis buffer may include from about 0.5 mM to about 5 mM Tris-EDTA and from about 10 mM to about 30 mM TCEP at a concentration of about 15× to about 25×.

As discussed above, the effective processing of a biological specimen may include one or more steps of thermal processing. Active temperature control of the mixing well may facilitate increased reaction and diffusion kinetics. The biological specimen and any chemical processing agents may be added to the mixing well and the biological specimen may be mixed by the mixing member. Since viscoelastic materials may have viscosity that depends upon the shear rate of the material, the mixing action of the mixing member may aid the processing by temporarily lowering the viscosity of the biological specimen. Prior to mixing, during mixing or after mixing, the temperature of the mixing well may be raised and/or lowered for thermal treatment of the biological specimen The thermal treatment may also promote cell lysis. During processing, the lysis micro-reactor may be heated to a temperature of at least about 60° C., at least about 75° C., at least about 90°C. or even at least about 100°C.

Thermal processing may take place by way of a holding device into which the mixing well may be placed that may provide both heat for thermal processing and the motor for the mixing structure. The holding device may include an opening for receiving the mixing well. The mixing well may be permanently attached to and/or integrally formed with the holding device. The mixing well may instead be removable from the holding device. As an example, a disposable mixing well may be removable from the holding device so that it is not necessary for the entire holding device to be disposable. Alternatively, the mixing well and holding device may both be disposable. The holding device may further include one or more conductive (e.g., highly thermally conductive) walls that contact the opening for receiving the mixing well. The one or more conductive walls may be composed of one or any combination of conductive materials including but not limited to silver, copper, aluminum, gold, brass, rhodium, platinum, titanium, highly thermally conductive polymer materials, or any combination thereof.

The holding device may also include a means for providing heat to the mixing well via the one or more conductive walls. The means for providing heat may be connected to a power source (e.g., a DC or AC power source) that provides electricity for heat production. The power source may be a battery located within the holding device or located external to the holding device. The power source may originate from an analysis and/or amplification device. The holding device may be powered by solar power. The means for providing heat may include thermoelectric devices, resistive heaters, power resistors, other types of heating devices or any combination thereof. The means for providing heat may also provide a cooling function to remove heat from the mixing well or any other portion of the processing device. Cooling may also be provided by a fan device.

The means for providing heat to the mixing well may include one or more temperature sensors for monitoring the temperature of the conductive walls, the mixing well, the biological specimen, or any combination thereof. The one or more temperature sensors may be in direct contact and/or thermal communication with a biological specimen. The one or more temperature sensors may include a resistance temperature device (RTD), thermistor, thermocouple, or infrared scanner. The one or more temperature sensors may be in direct contact with a wall that contacts a biological specimen. It may also be possible that the one or more temperature sensors may employ non-contact temperature detection (e.g., IR thermography). The means for providing heat to the mixing well may include a temperature control for raising and lowering temperature of the conductive walls, the mixing well, the biological specimen, or any combination thereof as required by any thermal treatment specifications. As an example, the temperature sensor may determine if the temperature of the mixing well and/or its contents should be raised or lowered to reach a starting temperature, an elevated temperature, a mucolytic temperature or a lysis temperature. A multitude of temperature set points and the times at each can be programmed. The temperature set points and times can be cycled through at least one heater and optional cooler to promote processes such as amplification of the biological target biological indicator. Alternatively, more than one chamber may be present for processing, each at its own isothermal set point and the fluid contents transferred among the chambers. The temperature sensor and temperature control may be integrated into one device that both controls and senses the temperature. The temperature sensor and temperature control may be separate devices. One or both of the temperature sensor and temperature control may be located within the holding device, or even within the mixing well. One or both of the temperature sensor and temperature control may be located external to the holding device but having a portion connected to the holding device for accurate temperature measurement and temperature control. The heaters and temperature sensors may take on a substantially cylindrical shape or any other shape that may minimize the space required for the heaters and sensors and/or maximize contact with one or more portions of the processing device.

The temperature control may require manual adjustment to the temperature or may be modified automatically according to a pre-programmed thermal treatment protocol. The thermal treatment protocol may be programmed via software that may be integrated within the holding device or may be part of a computing or control device located external from the holding device. The temperature control and/or thermal treatment protocol may be modified according to the composition of the biological specimen. For example, a biological specimen having a higher viscosity may require exposure to higher temperatures or exposure to greater number of variable temperatures in an effort to reduce the viscosity of the sample.

The processing device may also include a controller that is integrated with the processing device, separate from the processing device, or integrated with a separate amplification and/or detection device. The controller may be in communication with and may control thermal devices (e.g. resistive heaters, thermoelectric modules), motors and temperature sensors to operate the components of the processing device and perform a protocol input by a user via an interface. A central processing unit may be tasked with executing a predetermined protocol. One or more H-bridges may be useful for alternating the impeller direction or controlling the heating and/or cooling of any thermoelectric modules. Digital or analog outputs may be employed to turn on and/or turn off the motor, heaters, coolers and control the amount of voltage/current applied thereto. An analog-to-digital converter may be utilized in processing the signal from the temperature sensor. The controller may also include a display of the protocol status, including temperature, motor speed (e.g., torque), and progress may be displayed numerically and/or graphically by the display. It is possible that a bench-top instrument accompanies the processing device.

Several safety features may be built into the sample processing device. The main safety feature includes the separation of the sample from the device users. The processing device may be pre-sealed or enclosed and a cover, if any, may shut or seal tightly to minimize the chance of leakage. The cover itself may have a dual enclosure feature similar to well designed inflatable (e.g. a beach ball or an air mattress) where an outer cover seals tightly and an inner flap is sealed only when the sample is supposed to go through a transport path. Entry ports from the specimen container to the processing device may be sealed with heat and/or pressure to make a tight seal and to destroy potential chemical/biological hazards in the seated region. Automation of the processing steps may reduce the need for human interaction and potential human errors when handling the biological specimen and biological target biological indicator. The transport means may avoid the common use of centrifuges, thereby eliminating the risk of exposure in the rare but typically violent failure of the centrifuge. An optional ultraviolet light (typically in the 200 nm to 300 nm wavelength range) can be incorporated into the processing device to aid in destruction of any potential hazardous materials. The inexpensive and disposable nature of the processing device and mixers may allow for economical and safe disposal such as incineration and/or autoclave treatment of the processing device. Optional temperature sensitive paint, temperature sensitive wax, and/or a temperature film gauge can be applied to the outside of the processing device for quick visual inspection to ensure that the processing device has reached the proper temperature(s) during processing. Failsafe components may be included such as heaters that turn off automatically in the case of an equipment failure. Combined, these safety features may allow for minimal exposure of the user to any potentially hazardous contamination.

The amount of biological specimen that may be received from the specimen container into the processing device may be at least about 2 μl. The amount of biological specimen that may be received from the specimen container into the processing device may be less than about 4000 μl. The amount of biological specimen combined with chemical processing agents that may be received from the specimen container into the processing device may be from about 250 μl to about 2000 μl. The amount of biological specimen amplified may be the same as the amount received into the processing device or may be substantially less than the amount of biological specimen received into the processing device. As an example, the initial amount of biological specimen received by the lysis micro-reactor may be from about 200 μl to about 800 μl. The initial amount of biological specimen received by the lysis micro-reactor may be about 400 μl. Upon cell lysis and release of the target biological indicator, an aliquot of the mixing well contents may be transferred to an amplification well via a transfer means in an amount of only about 3 μl to about 50 μl. The amount of target biological indicator amplified may be less than about 40 μl less than about 30 μl, less than about 20 μl, or even less than about 10 μl.

After transfer of the biological specimen from the specimen container to the mixing well, the biological specimen may be treated mechanically, chemically and/or thermally as described herein. After treatment, the biological specimen or a portion of the biological specimen (e.g., any target biological indicator located on the capture strip) may be processed, amplified, detected, or any combination thereof. The amplification may allow for the detection of the presence or absence of particular genetic or disease related sequences. The amplification process may occur in the mixing well. In order to amplify only the target biological indicator, it may be necessary to remove any remaining biological specimen (e.g., waste material) from the mixing well. A wash step may be incorporated to further remove any remaining biological specimen. This removal may be performed by pumping the waste material from the mixing well into an additional well or elsewhere. As previously discussed, the pumping mechanism may be facilitated by the mixing member. The mixing member may spin in the opposite direction of that used for mixing (e.g., the mechanical treatment) for pumping purposes. The target biological indicator may be transferred from a first portion of the processing device to a second portion of the processing device that is spaced apart from the first portion but in fluid communication with the first portion.

As discussed herein, the amplification process may take place in a second location (e.g., the amplification portion). The amplification portion may be located within the processing device or may be located external from the processing device. The second location may be an internal amplification well, tube, path or channel located within the processing device. The second location may be an external amplification well located external from the processing device.

The processing device may also include a transport means for transferring at least a portion of the biological specimen to the amplification portion. The transport means may also facilitate the transfer of one or more substances throughout (e.g., within, into or out of) the processing device. The transport means may include a fluid transport path or tube. The transport means may include a capillary portion. The transport means may include a valve for controlling the transport function so that fluid flow may be stopped, slowed or otherwise controlled. The valve may be opened and/or closed automatically or manually.

The transport means include one or more channels or valves through which fluid and/or air returns back to the mixing portion. Thus the transport means, the mixing portion, or both may further include a pressure release portion to facilitate effective transport of biological specimen within the processing device. The processing device or a component of the processing device may include a means for introducing a pressure gradient so that a first portion of the processing device has a first pressure and a downstream portion of the processing device has a second pressure that is lower than the first pressure. As an example, the mixing well may be exposed to high pressure and the amplification well may be exposed to a lower pressure so that after biological specimen treatment the biological specimen or a portion of the biological specimen is moves from the high pressure area to the lower pressure area along a pressure gradient that facilitates the biological specimen movement. The transport means may include transport through a filter, chromatography column, hybridization area, or over any adherent material such as plastic, glass, at least one bead, and/or an optical microarray device in order to aid in trapping or purification of the biological target biological indicator. A filter may collect cell debris. The transport means may also include chemical reagents, media, probes, or the like.

It is possible that the transport means may include an amplification portion therein so that amplification occurs within the transport means. The biological specimen or a portion of the biological specimen (e.g., the target biological indicator) may be pumped from the mixing well through the transport means where it is amplified. Amplification and/or detection reagents necessary to carry out PCR and/or detection may be present during the amplification and thus may be pre-loaded in the amplification well or transferred thereto. The remaining biological specimen or portion of the biological specimen that undergoes amplification may then be pumped into a waste or collection well or tube located within the processing device or external to the processing device. Detection may be integrated into the well (e.g. real-time PCR). Alternatively, other detection methods (such as gel electrophoresis, probe hybridization, or the like) may be performed post-amplification external to or integrated into the processing device. In the event that amplification occurs within the transfer means, the means for providing heat may contact and/or provide heat to the transfer means so that the temperature of the biological specimen or portion of the biological specimen located within the transfer means can be raised and lowered for the amplification process. Further, the transfer means may be include a material that imparts flexibility to the transfer means so that the means can be compressed to minimize the profile width of the transfer means to improve the speed and accuracy of the amplification process.

The biological specimen or a portion of the biological specimen may be transferred to a PCR device (e.g., a PCR reaction chamber) such as that disclosed in U.S. application Ser. No. 12/918,594 fled Aug. 20, 2010 and U.S. Provisional Application No. 61/492,002, filed Jun. 1, 2011, both applications being incorporated by reference herein for all purposes. The amplification process described in the applications referenced above may include positioning some or all of the target biological indicator along with one or more PCR reagents between at least two or more opposing spaced apart thermocycling (e.g., thermoelectric) elements that operate by the Peltier effect in a PCR thermal cycling instrument. The PCR device disclosed therein in combination with the simultaneous treatment protocols of the present teachings may allow for effective diagnostic testing in less than 2 hours, more preferably less than 0.5 hours and even more preferably less than 0.2 hours.

Some or all of the lysis micro-reactor contents may be transferred to the PCR device manually (e.g., by pipette) or through a transport means such as that described above. Alternatively, the entire processing device or a portion of the processing device (e.g., the mixing well) may be located within the PCR device. The capture strip may be removed from the lysis micro-reactor and located into a PCR tube in which a thermocycling process takes place. The PCR may involve a thermocycling process where the temperature of the target biological indicator undergoes a series of temperature increases and decreases in an effort to amplify a desired nucleotide sequence.

The amplification and detection processes may involve any process including but not limited to polymerase chain reaction (PCR), reverse transcription polymerase chain reaction (RT-PCR), quantitative real time polymerase chain reaction (Q-PCR), gel electrophoresis, capillary electrophoresis, mass spectrometry, fluorescence defection, ultraviolet spectrometry, DNA hybridization, allele specific polymerase chain reaction, polymerase cycling assembly (PCA), asymmetric polymerase chain reaction, linear after the exponential polymerase chain reaction (LATE-PCR), helicase-dependent amplification (HDA), hot-start polymerase chain reaction, intersequence-specific polymerase chain reaction (ISSR), inverse polymerase chain reaction, ligation mediated polymerase chain reaction, methylation specific polymerase chain reaction (MSP), multiplex polymerase chain reaction, nested polymerase chain reaction, solid phase polymerase chain reaction, or any combination thereof.

The processing device may include a plurality of different wells and/or transfer means such that the type and arrangement of wells and transfer means may be tailored depending on the type of treatment and detection to be performed. The processing device may include one or more mixing wells, PCR wells, detection wells, water wells, reagent wells, waste wells, reverse transcriptase wells, washing wells, or the like. The processing device may also include one or more connecting channels in which a plurality of functions (filtering, hybridization, PCR, detection, or the like) may occur.

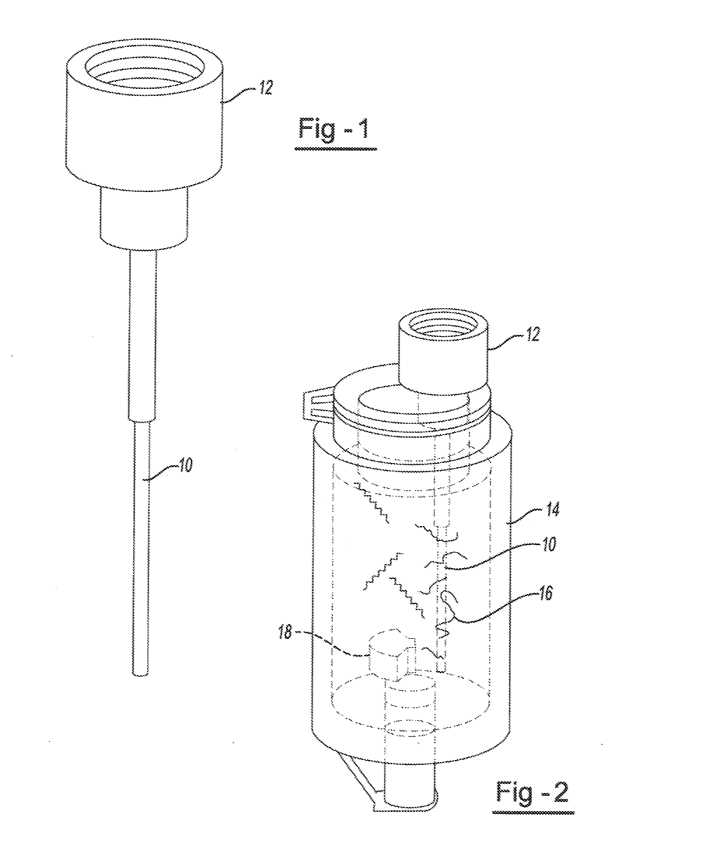

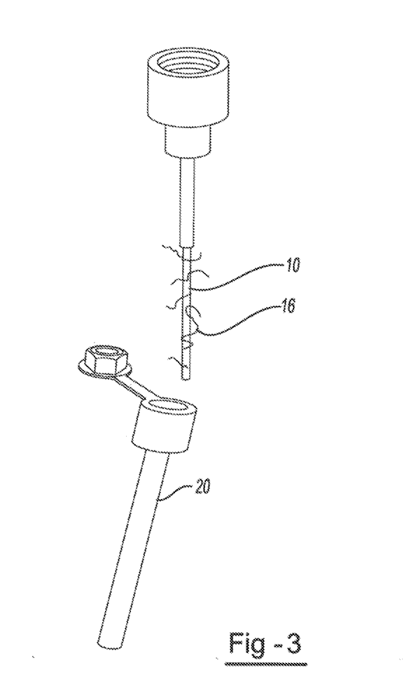

As shown for example in FIG. 1, a capture strip 10 may include a handling portion 12 that is located at one end of the capture strip. The handling portion 12 may remain external to the lysis micro-reactor 14 when the capture strip 10 is located into the lysis micro-reactor, as shown in FIG. 2. FIG. 2 further includes nucleic acids 16 within the lysis micro-reactor and located onto the capture strip 10. Also located within the lysis micro-reactor as shown is an impeller 18 for mixing the contents of the lysis micro-reactor.

After treatment of the biological sample within the micro-reactor, the capture strip 10 containing the nucleic acids 16 may be removed from the micro-reactor and located into a device 20 for further processing, as shown in FIG. 3. As described herein, this device may be a PCR cuvette.

The non-limiting examples below refer specifically to testing for C. difficile disease (CDI), however, testing of biological samples for other disease indicators is also envisioned by the methods described below.

Traditionally, diagnosis of CDI has been based on detecting the presence of C. difficile toxin A and/or B proteins in stool. The cytotoxin assay that detects toxin B-mediated cell cytotoxicity in stool, is very sensitive and produces accurate results but is time-consuming for routine use. Consequently, enzyme immunoassay (EIA) for C. difficile toxins in stool are widely used since they are easy to use and provide results within one day. However, the available EIA tests for one or both toxins are relatively insensitive, defecting only 30-70% of CDI-related disease. As a result, an EIA test for the C. difficile cell wall antigen glutamate dehydrogenase (GDH) has been developed having improved sensitivity but low specificity, as it detects both toxigenic and nontoxigenic strains. Therefore, EIA results for GDH or for toxins A or B may be inadequate when considered in isolation. Accordingly, a dual step assay that includes a GDH EIA test with a subsequent toxin EIA (either A, B, or both) has been developed. If both EIA assays are positive or negative, then the interpretation is straight-forward, but if the results are inconsistent (as is often the case), additional testing is required. The examples below demonstrate accuracy similar to that of the dual step assay, without the possibility of inconsistent results that require additional testing, such as loop-mediated isothermal DNA amplification, or LAMP testing which is both costly and time consuming.

### EXAMPLE 1

### Assay Sensitivity

Stool samples known to be negative for C. difficile (EIA negative and negative for GDH and toxin) were spiked with purified C. difficile DNA at varying concentrations. The samples were prepared to have DNA concentrations of 1.25 pg/ml (312 copies/ml), 0.125 pg/ml (31 copies/ml), and 0.025 pg/ml (8 copies/ml). 400 μl of each sample was added to an equivalent volume of lysis reagent in the lysis micro-reactor. A chelating resin powder (Chelex 100, available from Bio-Rad Laboratories, Hercules, Calif.) was added to the samples. The DNA was captured on polystyrene capture strips, washed, and transferred to PCR cuvettes. The electrophoresis results demonstrate that the assay can detect DNA concentrations as low as 31 copies/ml.

### EXAMPLE 2

### Comparative Testing with Dual EIA Screening

A total of 79 clinical samples from patients with suspected CDI were provided from the University of Nebraska Medical Center clinical laboratory and frozen at −20° C. until testing. Diarrheal stool samples were collected from patients at the request of the treating physician, and experiments described herein were performed on excess sample material. The de-identified samples were assigned a study number.

Dual EIA Screen—A dual EIA screening for both GDH and toxin A/B (Wampole™ C. DIFF QUIK CHEK COMPLETE® available from Techlab®, Inc., Blacksburg, Va.) was performed according to the manufacturer's instructions on all liquid stool samples submitted for evaluation of C. difficile presence. Samples with positive EIA results for both GDH and toxin A/B were considered truly positive, samples negative for both GDH and toxin A/B were considered truly negative, and no additional testing was necessary. For specimens with discordant GDH and toxin A/B results, LAMP (Illumigene C. difficile, available from Meridian Bioscience®, Inc., Cincinnati, Ohio) testing was performed. Among 79 EIA results, 12 samples were negative for both GDH and toxin A/B and 33 were positive for both GDH and toxin A/B, The remaining 34 samples were positive for GDH, but negative for toxin A/B. LAMP testing was performed only for clarification of results for the 34 EIA discordant samples. LAMP test results revealed 18 positive samples and 16 negative samples. Therefore of 79 total samples, 51 were positive and 28 were negative.

All 79 samples were re-tested using the protocol described below and thereafter compared to the dual EIA and LAMP results.

Lysis micro-reactor protocol—A lysis buffer of 20 mM TCEP (Tris[2-carboxyethyl]phosphine) and 20×TE (Tris-EDTA) was prepared. A wash buffer (TNTw) of 10 mM Tris (Tris[hydroxymethyl]aminomethane), 150 mM NaCl and 0.05% Tween 20 was prepared.

Clear polystyrene strips (0.127 mm×1 mm×4 mm) were sanded and incubated overnight in 20 mM EOC hydrochloride (N-[3-Dimethylaminopropyl]-N′-ethylcarbodiimide hydrochloride). The strips were washed once with the wash buffer (TNTw) then once with de-ionized water and stored in de-ionized water until use.

400 μl of thawed, unformed stool was mixed with 400 μl of the lysis buffer and transferred to a 1 ml capacity LMR (lysis micro-reactor). Dry Chelex 100 beads (Bio-Rad Laboratories, Inc., 40-80 mg) were added and a prepared capture strip was also placed in the lysis micro-reactor. The contents were heated to 92° C. and mixed for 5 minutes. The capture strip was removed from the micro-mixer and washed twice with distilled water before being placed in a cuvette containing 25 μl of previously prepared PCR mastermix. Each 25 μl reaction contained a final concentration of 0.2 mM dNTP's, 4 mM MgSO₄0.5 U KOD Hot Start DNA polymerase, 1×PCR Buffer for KOD Hot Start DNA polymerase (EMD Chemicals, Inc.): 0.4 mg/ml BSA (Ambion®, Inc.): 2 μM SYTO13 (Invitrogen™); 0.2 μM forward and reverse primers (as described below and and described by van den Berg et al. (2006)) (UNMC Eppley Molecular Biology Core Lab).

Amplification of a non-repeat region of the tcdB gene was performed using primers shown in Table 1 on a Philisa™ thermocycler (available from Streck, Inc., La Vista, Nebr.). The thermal protocol included an enzyme activation step at 95° C. for 30 seconds which was followed by 30 cycles of 95° C. for 3 seconds and 59° C. for 4 seconds and then by 15 cycles of 95° C. for 3 seconds, 59° C. for 7 seconds and 72° C. for 10 seconds. Gel electrophoresis was used for product detection.

TABLE 1
PCR primers

| Target | Length | Primer | Sequence |
|---|---|---|---|
| tcdB | 177 bp | Forward-398CLDs | GAAAGTTCAAGTTTACGCTC- |
| | | | AAT |
| | | Reverse-399CLDas | GCTGCACCTAAACTTACACCA |

Of the 79 samples tested using the capture strip and LMR, 23 tested demonstrated a negative test result and 56 demonstrated a positive result. Traditional testing using a combination of EIA, GDH/Toxin B and LAMP testing resulted in 28 negative results and 51 positive results. The use of the lysis micro-reactor and capture strips thus produced diagnostic results similar to those produced by the labor intensive multi-step dual EIA and LAMP tests.

Any numerical values recited herein include all values from the lower value to the upper value in increments of one unit provided that there is a separation of at least 2 units between any lower value and any higher value. As an example, if it is stated that the amount of a component or a value of a process variable such as, for example, temperature, pressure, time and the like is, for example, from 1 to 90, preferably from 20 to 80. more preferably from 30 to 70, it is intended that values such as 15 to 85, 22 to 68, 43 to 51, 30 to 32 etc. are expressly enumerated in this specification. For values which are less than one, one unit is considered to be 0.0001, 0.001, 0.01 or 0.1 as appropriate. These are only examples of what is specifically intended and all possible combinations of numerical values between the lowest value and the highest value enumerated are to be considered to be expressly stated in this application in a similar manner. As can be seen, the teaching of amounts expressed as “parts by weight” herein also contemplates the same ranges expressed in terms of percent by weight. Thus, an expression in the Detailed Description of the Invention of a range in terms of at “‘x’ parts by weight of the resulting polymeric blend composition” also contemplates a teaching of ranges of same recited amount of “x” in percent by weight of the resulting polymeric blend composition.”

Unless otherwise stated, all ranges include both endpoints and all numbers between the endpoints. The use of “about” or “approximately” in connection with a range applies to both ends of the range. Thus, “about 20 to 30” is intended to cover “about 20 to about 30”, inclusive of at least the specified endpoints.

The disclosures of all articles and references, including patent applications and publications, are incorporated by reference for ail purposes. The term “consisting essentially of” to describe a combination shall include the elements, ingredients, components or steps identified, and such other elements ingredients, components or steps that do not materially affect the basic and novel characteristics of the combination. The use of the terms “comprising” or “including” to describe combinations of elements, ingredients, components or steps herein also contemplates embodiments that consist essentially of the elements, ingredients, components or steps. By use of the term “may” herein, it is intended that any described attributes that “may” be included are optional.

Plural elements, ingredients, components or steps can be provided by a single integrated element, ingredient, component or step. Alternatively, a single integrated element, ingredient, component or step might be divided into separate plural elements, ingredients, components or steps. The disclosure of “a” or “one” to describe an element, ingredient, component or step is not intended to foreclose additional elements, ingredients, components or steps. All references herein to elements or metals belonging to a certain Group refer to the Periodic Table of the Elements published and copyrighted by CRC Press, Inc. 1989. Any reference to the Group or Groups shall be to the Group or Groups as reflected in this Periodic Table of the Elements using the IUPAC system for numbering groups.

It will be appreciated that concentrates or dilutions of the amounts recited herein may be employed. In general, the relative proportions of the ingredients recited will remain the same. Thus, by way of example, if the teachings call for 30 parts by weight of a Component A, and 10 parts by weight of a Component B, the skilled artisan will recognize that such teachings also constitute a teaching of the use of Component A and Component B in a relative ratio of 3:1. Teachings of concentrations in the examples may be varied within about 25% (or higher) of the stated values and similar results are expected. Moreover, such compositions of the examples may be employed successfully in the present methods.

It will be appreciated that the above is by way of illustration only. Other ingredients may be employed in any of the compositions disclosed herein, as desired, to achieve the desired resulting characteristics. Examples of other ingredients that may be employed include antibiotics, anesthetics, antihistamines, preservatives, surfactants, antioxidants, unconjugated bile acids, mold inhibitors, nucleic acids, pH adjusters, osmolarity adjusters, or any combination thereof.

It is understood that the above description is intended to be illustrative and not restrictive. Many embodiments as well as many applications besides the examples provided will be apparent to those of skill in the art upon reading the above description. The scope of the invention should, therefore, be determined not with reference to the above description, but should instead be determined with reference to the appended claims, along with the full scope of equivalents to which such claims are entitled. The disclosures of ail articles and references, including patent applications and publications, are incorporated by reference for all purposes. The omission in the following claims of any aspect of subject matter that is disclosed herein is not a disclaimer of such subject matter, nor should it be regarded that the inventors did not consider such subject matter to be part of the disclosed inventive subject matter.

1: A method for preparing a biological specimen comprising:
introducing a biological specimen into a processing device;
contacting the biological specimen with a lysis buffer to promote the release of one or more biological indicators;
treating the biological specimen mechanically within the processing device to cause sufficient turbulence to contact the one or more biological indicators within the biological specimen to a removable polystyrene adherent strip located within the processing device;

2. (canceled)

3. The method of claim 1, polymeric material is formed as a rectangle, or cylinder.

4. The method of claim 1, wherein the polystyrene adherent strip is located within the processing device while the biological specimen is treated.

5. (canceled)

6. The method of claim 1, wherein the lysis buffer includes Tris[2-carboxyethyl]phosphine.

7. The method of claim 1, wherein the polystyrene adherent strip is structurally pre-treated to improve the adhesion characteristics prior to contact with the biological specimen.

8. The method of claim 1, wherein the polystyrene adherent strip is transferred to a PCR cuvette after binding to one or more biological indicators.

9. The method of claim 1, wherein the polystyrene adherent strip binds to biological indicators associated with Clostridium difficile infection.

10. The method of claim 1, wherein the treatment of the biological specimen allows for diagnosis of positive for Clostridium difficile infection or negative for Clostridium difficile infection in less than 2 hours.

11. A method for preparing a biological specimen comprising:
introducing a biological specimen into a processing device;
contacting the biological specimen with a lysis buffer;
introducing a polystyrene adherent strip within the biological specimen;
mixing the biological specimen at a sufficient speed so that one or more biological indicators located within the specimen bind to the polystyrene adherent strip and remain located on the polystyrene adherent strip;
heating the -biological specimen to a temperature of at least about 90° C.;
transferring the polystyrene adherent strip and any biological indicators located thereon to a secondary device for additional processing.

12. The method of claim 11, wherein the additional processing includes a polymerase chain reaction processing step that effectively amplifies target DNA in less than 0.5 hours.

13. The method of claim 11, wherein the polystyrene adherent strip binds to biological indicators associated with Clostridium difficile infection.

14. The method of claim 11, wherein the lysis buffer includes Tris[2-carboxyethyl]phosphine and Tris-EDTA.

15. The method of claim 1, wherein the mixing is facilitated by agitation of the specimen within the processing device.

16. The method of claim 11, wherein at least a portion of the polystyrene adherent strip is located substantially parallel to the vertical axis of the processing device.

17. The method of claim 11, wherein chelating beads are added to the biological specimen during processing.

18. A method for preparing a biological specimen comprising:
introducing a biological specimen into a processing device;
contacting the biological specimen with a lysis buffer including Tris[2-carboxyethyl]phosphine and Tris-EDTA;
contacting the biological specimen with chelating beads;
providing a structurally pre-treated polystyrene adherent strip;
locating the polystyrene adherent strip within the processing device so that at least a portion of the polystyrene adherent strip is arranged substantially parallel to the vertical axis of the processing device;
mixing the biological specimen at a sufficient speed so that any biological indicators boated within the specimen bind to the polystyrene adherent strip and remain located on the polystyrene adherent strip;
heating the biological specimen to a temperature of at least about 90° C.;
transferring the polystyrene adherent strip and any biological indicators located thereon to a secondary device for additional processing.

19. The method of claim 18, wherein the biological indicators are associated with Clostridium difficile infection.

20. The method of claim 18, wherein the treatment of the biological specimen allows for a diagnosis of positive for Clostridium difficile infection or negative for Clostridium difficile infection in less than 2 hours.

21. The method of claim 18, wherein the structural pre-treatment of the polystyrene adherent strip includes a step of texturizing the polystyrene adherent strip.

22. The method of claim 18, wherein the polystyrene adherent strip is injection molded.
